(19) United States
(12) Patent Application Publication
(10) Pub. No.: US 2008/0138282 A1
(43) Pub. Date: Jun. 12, 2008

(54) Radiolabeled Arylsulfonyl Compounds and Uses Thereof

(75) Inventors: Joseph John Mann, Riverdale, NY (US); J.S. Dileep Kumar, Fort Lee, NJ (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 11/597,866

(22) PCT Filed: May 18, 2005
(86) PCT No.: PCT/US05/17529

§ 371 Date: Feb. 29, 2008

Related U.S. Application Data
(60) Provisional application No. 60/577,108, filed on Jun. 3, 2004.

(51) Int. Cl. A61K 51/04; A61K 101/02

(52) U.S. Cl. 424/1.89

(57) ABSTRACT

The present invention relates to Radiolabeled Arylsulfonyl Compounds and methods of use thereof as imaging agents for the COX-2 enzyme using positronemission tomograpy (PET). Methods of making the Radiolabeled Arylsulfonyl Compounds and pharmaceutical compositions comprising an effective amount of a Radiolabeled Arylsulfonyl Compound are also disclosed.

### FIELD OF THE INVENTION

The present invention relates to Radiolabeled Arylsulfonyl Compounds and methods of use thereof as imaging agents for the COX-2 enzyme using positron-emission tomograpy (PET). Methods of making the Radiolabeled Arylsulfonyl Compounds and pharmaceutical compositions comprising an effective amount of a Radiolabeled Arylsulfonyl Compound are also disclosed.

### BACKGROUND OF THE INVENTION

Cyclooxygenase (COX) is an enzyme required for the conversion of arachidonic acid to prostaglandins. Prostaglandins effect a diverse variety of physiological functions, such as gastrointestinal functions, renal homeostasis, uterine contraction, embryo implantation, modulation of blood pressure, lowering of progesterone levels, platelet aggregation and regulation of body temperature. There are three known isoforms of COX: COX-1, COX-2, and COX-3. Among these isoforms, COX-1 is predominantly constitutive, and is found in most tissues, particularly in platelets, stomach and kidney. COX-2 is predominantly inducible, though it is also constitutive in kidney, brain, heart, liver, testicles and tracheal epithelia. COX-2 is responsible for the biosynthesis of inflammatory prostaglandins and the levels of COX-2 can increase ten to twenty fold in inflammation, particularly in macrophages, monocytes, synoviocytes, chondrocytes, fibroblasts and endothelial cells. While the structures of these two enzymes are mostly similar, they also differ in a number of ways. COX-2 is more rapidly degraded, has a shorter half-life and possesses a larger binding site due to a secondary internal pocket. Compounds binding to this secondary pocket selectively inhibit COX-2. The third isoform, COX-3 has been recently identified and is believed to be the isoform responsible for the antipyretic and analgesic activities of NSAIDs.

Non steroidal anti-inflammatory drugs (NSAIDs) have potent analgesic and anti-inflammatory activity, which is believed to be due to the inhibition of COX-2. The therapeutic effects of NSAIDs, however, are counterbalanced by the presence of gastrointestinal side effects, which are thought to result from the inhibition of the COX-1 isoform. Since the discovery of the COX-2 isoform in 1991, rapid progress has been made in the development of COX-2 selective inhibitors (COXIBs), which maintain the therapeutic benefits of NSAIDs but have greatly diminished gastrointestinal side effects. To date, three selective COX-2 inhibitors, Celebrex®, Vioxx® and Bextra® have been approved for the treatment of arthritis and pain.

Several studies have suggested that COX-2 overexpression contributes to the pathogenesis of inflammation, arthritis, cancer, ulcers, pain sensation, neuropsychiatric disorders, and neurodegenerative diseases such as stroke, Alzheimer's disease and Parkinson's disease. Elevation of COX-2 levels is believed to be involved in the inflammatory response and non-steroidal inhibitors of the enzyme are potent anti-inflammatory agents.

Despite current knowledge regarding the pathology and treatment of inflammatory diseases, there is a lack of specific imaging agents that can successfully image inflammation. Several radiological techniques, including computed tomography, magnetic resonance imaging and ultrasonography have been utilized to image inflammation. But these techniques rely on anatomical changes and are not able to detect the early stages of inflammation due to the lack of substantial anatomical changes during the beginning of an inflammatory process. Positron emission tomography (PET) is a dynamic, non-invasive imaging technique used in nuclear medicine to study various biochemical and biological process in vivo, and like other dynamic imaging protocols, has the ability collect images repeatedly over time and provide information about regional distribution of the tracer as well as the change in compartmental distribution as a function of time. As such, PET lends itself directly to measuring kinetic processes, such as rate of tracer uptake by cells, substrate metabolic rates, receptor density/affinity, and regional blood flow. Labeled compounds can be administered in nanomolar or picomolar concentrations and allowing imaging studies to be performed without perturbing the biological system being studied.

To help more completely understand the roles of COX-2, it would be of great benefit to non-invasively and quantitatively monitor COX-2 expression in vivo. Highly selective COX-2 inhibitors are therefore promising candidates for the development of PET imaging probes for COX-2 expression.

Presently there are no reported PET ligands that are selective for the COX-2 enzyme and which have provided useful images in living brain and body. The synthesis of [¹⁸F] and [¹¹C] analogues of COX-2 inhibitors has been reported, but until now, no validating images have been reported for these respective PET probes. The radiosynthesis of [¹⁸F]-SC58125, a less potent COX-2 inhibitor, has also been reported as a COX-2 PET tracer. This compound however, suffers from de-[¹⁸F]fluorination and high non-specific binding which prevent it being a useful probe for PET imaging. The [¹⁸F] labeling of the COX-2 selective inhibitors DuP-697 and desbromoDuP-697 has been reported. The in vivo evaluation of these ligands shows an unusual regional pattern of COX-2 expression that is not in agreement with the reported COX-2 distribution, thus making the use of this ligand questionable for the in vivo quantification of COX-2. More recently, the synthesis of several [¹⁸F]-labeled COX-2 inhibitors were reported using a bromine to [¹⁸F]fluorine displacement and a [¹⁸F]fluorine for trialkylammonium triflate exchange reaction.

Thus, there remains a need in the art for COX-2 selective PET tracers which are useful for imaging COX-2 expression. The present invention addresses this need.

### SUMMARY OF THE INVENTION

This invention relates to compounds of Formula (I) and I(a) (the “Radiolabeled Arylsulfonyl Compounds”) which are useful as positron emission tomography (PET) imaging agents for the detection of COX-2 protein expression in a subject, and to monitor the progress or regression of an inflammatory disease in a subject. The invention also relates to methods of making the Radiolabeled Arylsulfonyl Compounds.

In one aspect, the present invention provides a method for detecting in vivo COX-2 protein expression in a subject, the method comprising the steps:

(a) administering to the subject an imaging-effective amount of a compound having the formula:

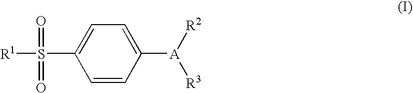

or a pharmaceutically acceptable salt thereof,
wherein:

A is -aryl, —C₃-C₇ cycloalkyl, —C₃-C₇ cycloalkenyl, or -3- to 7-membered heterocycle;

R¹ is a ¹¹C-labeled C₁-C₆ alkyl group, —¹⁸F-labeled C₁-C₆ alkyl group or —³H-labeled C₁-C₆ alkyl group;

R² is -aryl, —C₃-C₇ cycloalkyl, —C₃-C₇ cycloalkenyl, or -3- to 7-membered heterocycle, each of which may be unsubstituted or independently substituted with one or more -halo, —CF₃, —C₁-C₆ alkyl, —C₁-C₆ alkenyl, —(C₁-C₆ alkylene)-aryl, —C₁-C₆ alkynyl, —N(R⁴)₂, —CN, —OR⁴, —SR⁴, —S(O)—R⁴, —SO₂—R⁴, —SO₂NH—R⁴, —SO₃H, —NH—SO₂—R⁴, —C(O)R⁵ or —NHC(O)R⁵ groups.

R³ is —H, -halo, —CF₃, —C₁-C₆ alkyl, —C₁-C₆ alkenyl, -aryl, —(C₁-C₆ alkylene)-aryl, —C₁-C₆ alkynyl, —C₃-C₇ cycloalkyl, —C₃-C₇ cycloalkenyl, -3- to 7-membered heterocycle, —N(R⁴)₂, —CN, —OR⁴, —SR⁴, —S(O)—R⁴, —SO₂—R⁴, —SO₂NH—R⁴, —SO₃H, —NH—SO₂—R⁴, —C(O)R⁵ or —NHC(O)R⁵, wherein a —C₃-C₇ cycloalkyl, —C₃-C₇ cycloalkenyl, -3- to 7-membered heterocycle, or -aryl group may be unsubstituted or independently substituted with one or more -halo, —CF₃, —C₁-C₆ alkyl, —C₁-C₆ alkenyl, —(C₁-C₆ alkylene)-aryl, —C₁-C₆ alkynyl, —N(R⁴)₂, —CN, —OR⁴, —SR⁴, —S(O)—R⁴, —SO₂—R⁴, —SO₂NH—R⁴, —SO₃H, —NH—SO₂—R⁴, —C(O)R⁵ or —NHC(O)R⁵ groups;

each R⁴ is independently —H, —C₁-C₆ alkyl, —C₁-C₆ alkenyl, —C₁-C₆ alkynyl, -aryl, —(C₁-C₆ alkylene)-aryl, —C₃-C₇ cycloalkyl, —C₃-C₇ cycloalkenyl or -3- to 7-membered heterocycle; and

R⁵ is —R⁴, —N(R⁴)₂ or —OR⁴; and

(b) detecting the radioactive emission of the compound administered in step (a).

In the present methods, the radioactive emissions from ¹¹C and ¹⁸F can be detected using positron emission tomography, and the ³H radioactive emission can be detected using autoradiography for imaging COX-2 protein expression in a subject. The radioactive emission can be detected anywhere in the body of the subject. In one embodiment, the radioactive emission is detected in the brain, joints, heart, kidney or any combination thereof, of the subject. In a further embodiment, the subject can be known or suspected to have one or more of the following conditions: inflammation, arthritis, a neoplastic disease, Alzheimer's disease, Parkinson's disease, atherosclerosis, stroke, myocardial infarction, diabetes, allograft rejection, urogenital disease, cancer, central nervous system disorders, brain injury and renal disorders.

In another aspect, the invention provides methods for making Radiolabeled Arylsulfonyl Compounds having the formula:

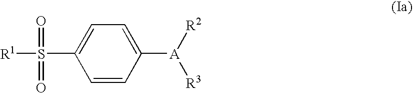

wherein:

A is -aryl, —C₃-C₇ cycloalkyl, —C₃-C₇ cycloalkenyl, or -3- to 7-membered heterocycle;

R¹ is a ¹¹C-labeled C₁-C₆ alkyl group;

R² is -aryl, —C₃-C₇ cycloalkyl, —C₃-C₇ cycloalkenyl, or -3- to 7-membered heterocycle, each of which may be unsubstituted or independently substituted with one or more -halo, —CF₃, —C₁-C₆ alkyl, —C₁-C₆ alkenyl, —(C₁-C₆ alkylene)-aryl, —C₁-C₆ alkynyl, —N(R⁴)₂, —CN, —OR⁴, —SR⁴, —S(O)—R⁴, —SO₂—R⁴, —SO₂NH—R⁴, —SO₃H, —NH—SO₂—R⁴, —C(O)R⁵ or —NHC(O)R⁵ groups.

R³ is —H, -halo, —CF₃, —C₁-C₆ alkyl, —C₁-C₆ alkenyl, -aryl, —(C₁-C₆ alkylene)-aryl, —C₁-C₆ alkynyl, —C₃-C₇ cycloalkyl, —C₃-C₇ cycloalkenyl, -3- to 7-membered heterocycle, —N(R⁴)₂, —CN, —OR⁴, —SR⁴, —S(O)—R⁴, —SO₂—R⁴, —SO₂NH—R⁴, —SO₃H, —NH—SO₂—R⁴, —C(O)R⁵ or —NHC(O)R⁵, wherein a —C₃-C₇ cycloalkyl, —C₃-C₇ cycloalkenyl, -3- to 7-membered heterocycle, or -aryl group may be unsubstituted or independently substituted with one or more -halo, —CF₃, —C₁-C₆ alkyl, —C₁-C₆ alkenyl, —(C₁-C₆ alkylene)-aryl, —C₁-C₆ alkynyl, —N(R⁴)₂, —CN, —OR⁴, —SR⁴, —S(O)—R⁴, —SO₂—R⁴, —SO₂NH—R⁴, —SO₃H, —NH—SO₂—R⁴, —C(O)R⁵ or —NHC(O)R⁵ groups;

each R⁴ is independently —H, —C₁-C₆ alkyl, C₁-C₆ alkenyl, —C₁-C₆ alkynyl, -aryl, —(C₁-C₆ alkylene)-aryl, —C₃-C₇ cycloalkyl, —C₃-C₇ cycloalkenyl or -3- to 7-membered heterocycle; and

R⁵ is —R⁴, —N(R⁴)₂ or —OR⁴,

the method comprising the steps:

(a) contacting a compound having the formula:

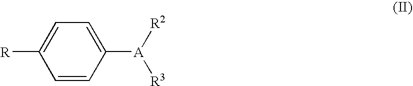

wherein:

R is —SH or —SC(O)—(CH₂)₂CH₃; and A, R² and R³ are as defined above for the compounds of formula (Ia),

with a base for a time and at a temperature sufficient to make an intermediate having the Formula (III):

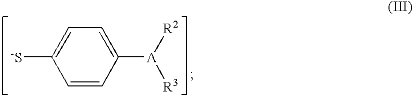

(b) contacting the intermediate of Formula (III) with a compound having the formula R¹X for a time and at a temperature sufficient to make a compound of Formula (IV):

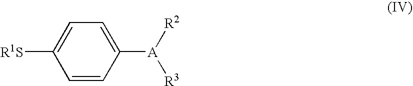

wherein R¹ is a ¹¹C-labeled C₁-C₆ alkyl, X is a leaving group and A, R² and R³ are as defined above for the compounds of Formula (Ia); and

(c) contacting the compound of Formula (IV) with an oxidizing agent for a time and at a temperature sufficient to make a compound having the Formula (Ia):

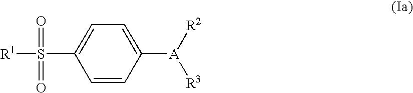

wherein A, R¹, R² and R³ are as defined above for the Compounds of Formula (Ia).

### BRIEF DESCRIPTION OF THE FIGURES

FIG. 1 shows three currently marketed COX-2 Selective Inhibitors (COXIBs).

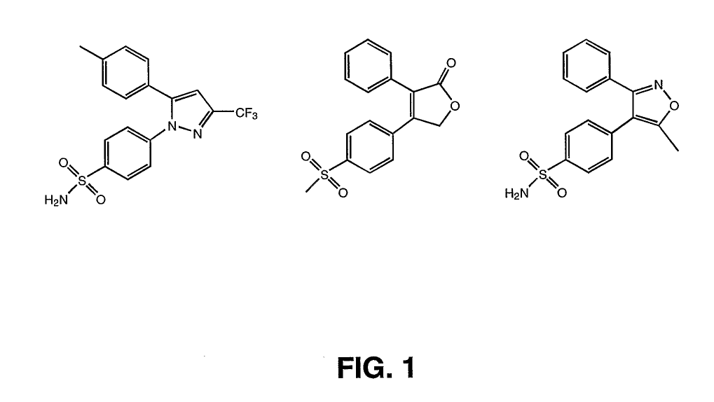

FIG. 2 shows two previously reported radiolabeled COXIBs, where the radiolabel is not part of a methylsulfonyl group, as opposed to certain embodiments of the present invention.

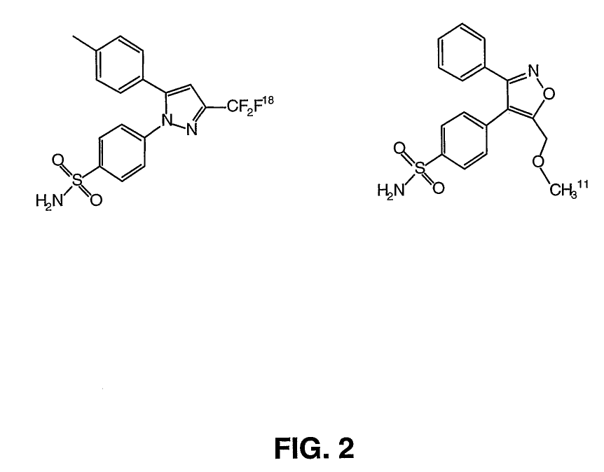

FIG. 3 shows three COXIBs that can be radiolabeled using the methods of the present invention.

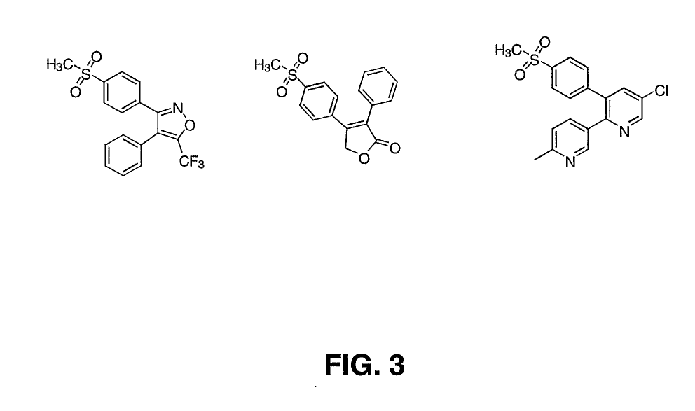

### DETAILED DESCRIPTION OF THE INVENTION

### Definitions and Abbreviations

The terms used herein having following meaning:

The term “—C₁-C₆ alkyl” as used herein, refers to a straight chain or branched non-cyclic hydrocarbon having from 1 to 6 carbon atoms, wherein one of the hydrocarbon's hydrogen atoms has been replaced with a single bond. Representative straight chain —C₁-C₆ alkyls include -methyl, -ethyl, -n-propyl, -n-butyl, -n-pentyl, and -n-hexyl. Representative branched —C₁-C₆ alkyls include -isopropyl, -sec-butyl, -isobutyl, -tert-butyl, -isopentyl, -neopentyl, 1-methylbutyl, 2-methylbutyl, 3-methylbutyl, 1,1-dimethylpropyl, 1,2-dimethylpropyl, 1-methylpentyl, 2-methylpentyl, 3-methylpentyl, 4-methylpentyl, 1-ethylbutyl, 2-ethylbutyl, 3-ethylbutyl, 1,1-dimethylbutyl, 1,2-dimethylbutyl, 1,3-dimethylbutyl, 2,2-dimethylbutyl, 2,3-dimethylbutyl, 3,3-dimethylbutyl, -isopropyl, -sec-butyl, -isobutyl, -neohexyl, -isohexyl, and the like. In one embodiment, the C₁-C₆ alkyl is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl.

A “¹¹C-labeled C₁-C₆ alkyl group” is a C₁-C₆ alkyl group, as defined above, wherein one of the C₁-C₆ alkyl group's carbon atoms has been replaced with a ¹¹C atom. Representative ¹¹C-labeled C₁-C₆ alkyls include, but are not limited to —¹¹CH₃, —CH₂¹¹CH₃, —CH₂CH₂¹¹CH₃, —CH₂CH₂CH₂¹¹CH₃, —CH₂CH₂CH₂CH₂¹¹CH₃, and —CH₂CH₂CH₂CH₂CH₂¹¹CH₃.

A “¹⁸F-labeled C₁-C₆ alkyl group” is a C₁-C₆ alkyl group, as defined above, wherein one of the C₁-C₆ alkyl group's hydrogen atoms has been replaced with a ¹⁸F atom. Representative ¹⁸F-labeled C₁-C₆ alkyls include, but are not limited to —CH₂¹⁸F, —CH₂CH₂¹⁸F, —CH₂CH₂CH₂¹⁸F, —CH₂CH₂CH₂CH₂¹⁸F, —CH₂CH₂CH₂CH₂CH₂¹⁸F, and —CH₂CH₂CH₂CH₂CH₂CH₂¹⁸F.

A “³H-labeled C₁-C₆ alkyl group” is a C₁-C₆ alkyl group, as defined above, wherein one of the C₁-C₆ alkyl group's hydrogen atoms has been replaced with a ³H atom. Representative ³H-labeled C₁-C₆ alkyls include, but are not limited to —CH₂³H, —CH₂CH₂³H, —CH₂CH₂CH₂³H, —CH₂CH₂CH₂CH₂³H, —CH₂CH₂CH₂CH₂CH₂³H, and —CH₂CH₂CH₂CH₂CH₂CH₂³H.

The term “C₂-C₆ alkenyl” as used herein, refers to a straight chain or branched non-cyclic hydrocarbon having from 2 to 6 carbon atoms and including at least one carbon-carbon double bond, wherein one of the hydrocarbon's hydrogen atoms has been replaced with a single bond. Representative straight chain and branched C₂-C₆ alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl, and the like. In one embodiment, the C₂-C₆ alkenyl is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl.

The term “C₂-C₆ alkynyl” as used herein, refers to a straight chain or branched non-cyclic hydrocarbon having from 2 to 6 carbon atoms and including at lease one carbon-carbon triple bond, wherein one of the hydrocarbon's hydrogen atoms has been replaced with a single bond. Representative straight chain and branched C₂-C₆ alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl, and the like. In one embodiment, the C₂-C₆ alkynyl is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl.

The term “C₁-C₆ alkylene” as used herein, refers to a straight chain or branched non-cyclic hydrocarbon having from 1 to 6 carbon atoms, wherein two of the hydrocarbon's hydrogen atoms have been replaced with single bonds.

The term “aryl” as used herein refers to a phenyl group, a biphenyl group or a naphthyl group. In one embodiment, the aryl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl.

The term “C₃-C₇ monocyclic cycloalkyl” as used herein is a 3-, 4-, 5-, 6- or 7-membered saturated non-aromatic monocyclic cycloalkyl ring. Representative C₃-C₇ monocyclic cycloalkyl groups include, but are not limited to, cyclopropyl, cyclobutyl, cyclopentyl, cyclohexyl and cycloheptyl. In one embodiment, the C₃-C₇ monocyclic cycloalkyl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl.

The term “C₃-C₇ monocyclic cycloalkenyl” as used herein is a 3-, 4-, 5-, 6- or 7-membered non-aromatic monocyclic carbocyclic ring having at least one endocyclic double bond, but which is not aromatic. It is to be understood that when any two groups, together with the carbon atom to which they are attached form a C₃-C₇ monocyclic cycloalkenyl group, the carbon atom to which the two groups are attached remain tetravalent. Representative C₃-C₇ monocyclic cycloalkenyl groups include, but are not limited to, cyclopropenyl, cyclobutenyl, 1,3-cyclobutadienyl, cyclopentenyl, 1,3-cyclopentadienyl, cyclohexenyl, 1,3-cyclohexadienyl, cycloheptenyl, 1,3-cycloheptadienyl, 1,4-cycloheptadienyl and -1,3,5-cycloheptatrienyl. In one embodiment, the C₃-C₇ monocyclic cycloalkenyl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl.

The term “halo” as used herein, refers to —F, —Cl, —Br, or —I.

The term “subject,” as used herein, includes, but is not limited to, a non-human animal, such as a cow, monkey, horse, sheep, pig, chicken, turkey, quail, cat, dog, mouse, rat, rabbit, or guinea pig; and a human. In one embodiment, a subject is a human.

The term “3- to 7-membered heterocycle” refers to: (i) a 3- or 4-membered non-aromatic monocyclic cycloalkyl in which 1 of the ring carbon atoms has been replaced with a N, O or S atom; (ii) a 5-, 6-, or 7-membered aromatic or non-aromatic monocyclic cycloalkyl in which 1-4 of the ring carbon atoms have been independently replaced with a N, O or S atom. The term 3- to 7-membered heterocycle also encompasses any heterocycles described by (i) or (ii) which are fused to a benzene ring, or in which any one of the ring carbon atoms comprises a carbonyl group, such as in lactam and lactone ring systems. The non-aromatic 3- to 7-membered heterocycles can be attached via a ring nitrogen, sulfur, or carbon atom. The aromatic 3- to 7-membered heterocycles are attached via a ring carbon atom. Representative examples of a 3- to 7-membered heterocycle group include, but are not limited to, dihydrofuran-2-one, dihydrofuranyl, furanyl, benzofuranyl, furazanyl, imidazolidinyl, imidazolinyl, imidazolyl, benzimidazolyl, indazolyl, indolinlyl, indolyl, indolizinyl, isoindolinyl, isothiazolyl, isoxazolyl, benzisoxazolyl, morpholinyl, oxadiazolyl, oxazolidinyl, oxazolyl, benzoxazolyl, oxazolidinyl, pyrimidinyl, phenanthridinyl, phenanthrolinyl, piperazinyl, piperidinyl, pyranyl, benzopyranyl, pyrazinyl, pyrazolidinyl, pyrazolinyl, pyrazolyl, pyridazinyl, pyridooxazole, pyridoimidazole, pyridothiazole, pyridyl, pyrimidinyl, pyrrolidinyl, pyrrolinyl, quinolinyl, isoquinolinyl, quinoxalinyl, phthalazinyl, cinnolinyl, quinolizinyl, quinazolinyl, quinuclidinyl, tetrahydrofuranyl, thiadiazinyl, thiadiazolyl, thiazolyl, benzthiazolyl, thienyl, thienothiazolyl, thienooxazolyl, thienoimidazolyl, thiomorpholinyl, thiophenyl, benzothiphenyl, triazinyl, and triazolyl. In one embodiment, the 3- to 7-membered heterocycle group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl.

When a first group is “substituted with one or more” second groups, each of one or more of the first group's hydrogen atoms is replaced with a second group. In one embodiment each carbon atom of a first group is independently substituted with one or two second groups. In another embodiment each carbon atom of a first group is independently substituted with only one second group.

The term “COXIB” as used herein, refers to a compound which is selectively binds the COX-2 enzyme and inhibits enzyme function.

As used herein, a “COX-2 selective agent” refers to a compound that can selectively interact with the COX-2 protein relative to the other COX isoforms. COX-2 selective agents includes compounds that specifically bind to COX-2 and inhibit enzyme function, i.e., COXIBs.

The term “imaging-effective amount” when used in connection with a Radiolabeled Arylsulfonyl Compound, is an amount of the compound that is sufficient to produce a visible image when the compound is administered to a subject and the radiation emitted by the compound is detected using positron-emission tomography (“PET”).

The phrase “pharmaceutically acceptable salt,” as used herein, is a salt of an acid and a basic nitrogen group of a Radiolabeled Arylsulfonyl Compound. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. The term “pharmaceutically acceptable salt” also refers to a salt of a Radiolabeled Arylsulfonyl Compound having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like. The term “pharmaceutically acceptable salt” also includes a hydrate of a Radiolabeled Arylsulfonyl Compound.

The term “isolated” as used herein means separate from other components of a reaction mixture or natural source. In certain embodiments, the isolate contains at least 30%, at least 35%, at least 40%, at least 45%, at least 50%, at least 55%, at least 60%, at least 65%, at least 70%, at least 75%, at least 80%, at least 85%, at least 90%, at least 95% or at least 98% of a Radiolabeled Arylsulfonyl Compound by weight of the isolate. In one embodiment, the isolate contains at least 95% of a Radiolabeled Arylsulfonyl Compound by weight of the isolate.

The following abbreviations are used herein and have the indicated definitions: DMF is N,N-dimethylformamide, 2,6-lutidine is 2,6-dimethylpyridine, mCPBA is m-chloroperoxybenzoic acid, MeOH is methanol; MS is mass spectrometry, NMR is nuclear magnetic resonance, Oxone® is a potassium peroxymonosulfate formulation (Du Pont, Wilmington, Del.), TBAOH is tetrabutylammonium hydroxide, TFAA is trifluoroacetic anhydride, and THF is tetrahydrofuran.

### The Radiolabeled Arylsulfonyl Compounds

The Radiolabeled Arylsulfonyl Compounds are useful as imaging agents for the COX-2 enzyme.

In certain embodiments, the Radiolabeled Arylsulfonyl Compound have one or more of the following characteristics: (i) high affinity and selectivity for the COX-2 isoform compared to the COX-1 and COX-3 isoforms; (ii) sufficient lipophilicity to allow rapid blood-brain-barrier penetration and generation of polar metabolites that do not cross the blood-brain-barrier; and (iii) high specific activity of the radioligand group R¹.

It is possible for the Radiolabeled Arylsulfonyl Compounds to have one or more chiral centers and as such the Radiolabeled Arylsulfonyl Compounds can exist in various stereoisomeric forms. Accordingly, Formula (I), although not depicting specific stereoisomers of the Radiolabeled Arylsulfonyl Compounds, are understood to encompass all possible stereoisomers.

The Radiolabeled Arylsulfonyl Compounds may be described by chemical name, chemical structure, or both. In any instance where both a chemical name and a chemical structure are provided, it is understood that the structural description takes precedence.

### The Radiolabeled Arylsulfonyl Compounds of Formula (I)

As stated above, the present invention encompasses Radiolabeled Arylsulfonyl Compounds having the Formula (I):

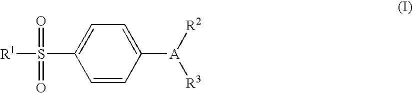

or a pharmaceutically acceptable salts thereof, wherein A, R¹, R² and R³ are as defined above for the Radiolabeled Arylsulfonyl Compounds of Formula (I).

In one embodiment A is -aryl or -3 to 7-membered heterocycle.

In another embodiment, A is phenyl, isoxazolyl, pyridyl or dihydrofuran-2-one.

In one embodiment, R¹ is a ¹¹C-labeled C₁-C₆ alkyl group.

In one embodiment, R¹ is a ¹⁸F-labeled C₁-C₆ alkyl group.

In one embodiment, R¹ is a ³H-labeled C₁-C₆ alkyl group.

In a specific embodiment, R¹ is —¹¹CH₃.

In one embodiment, R² is -aryl or -3 to 7-membered heterocycle.

In another embodiment, R² is phenyl or pyridyl.

In one embodiment, R³ is H, halo, C₁-C₆ alkyl or CF₃.

Illustrative Radiolabeled Arylsulfonyl Compounds of Formula (I) include the compounds listed below:

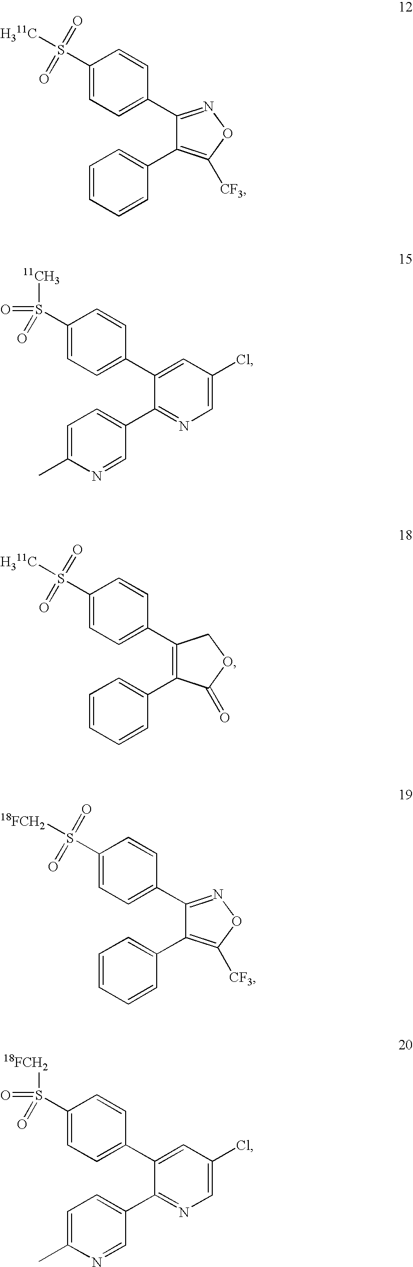

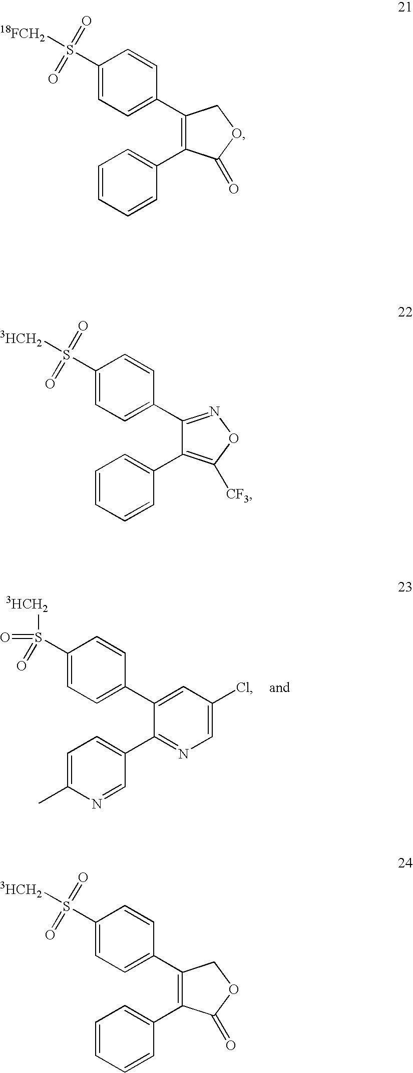

and pharmaceutically acceptable salts thereof.

### The Radiolabeled Arylsulfonyl Compounds of Formula (Ia)

As stated above, the present invention encompasses Radiolabeled Arylsulfonyl Compounds having the Formula (Ia):

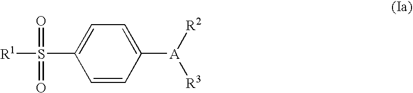

or pharmaceutically acceptable salts thereof, wherein A, R¹, R² and R³ are as defined above for the Radiolabeled Arylsulfonyl Compounds of Formula (Ia).

In one embodiment A is -aryl or -3 to 7-membered heterocycle.

In another embodiment, A is -phenyl, -isoxazolyl, -pyridyl or -dihydrofuran-2-one.

In one embodiment, R¹ is —¹¹CH₃.

In one embodiment, R² is -aryl or -3 to 7-membered heterocycle.

In another embodiment, R² is -phenyl or -pyridyl.

In one embodiment, R³ is —H, -halo, —C₁-C₆ alkyl or —CF₃.

Illustrative Radiolabeled Arylsulfonyl Compounds of Formula (I) include the compounds listed below:

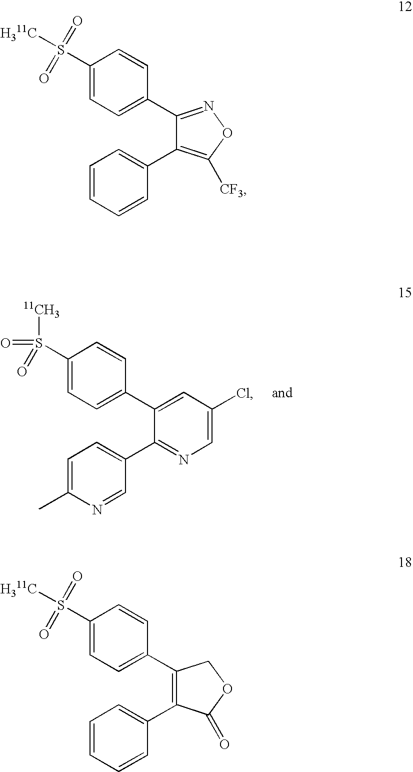

and pharmaceutically acceptable salts thereof.

### Methods for Making the Radiolabeled Arylsulfonyl Compounds

The Radiolabeled Arylsulfonyl Compounds can be made using the synthetic procedures outlined below in Schemes 1-4.

Scheme 1 shows methods for making compounds of Formula 3, which are useful intermediates for making the Radiolabeled Arylsulfonyl Compounds of Formula (I).

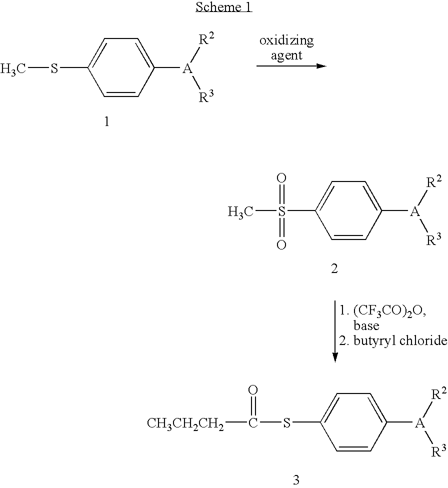

wherein A, R² and R³ are defined above for the Compounds of Formula (I).

The phenyl methyl thio compounds of Formula 1 can be oxidized using an oxidizing agent, such as mCPBA, to provide the phenyl methyl sulfoxide compounds of Formula 2. The compounds of Formula 2 can subsequently be reacted with trifluoroacetic anhydride in the presence of a base, such as 2,6-lutidine, followed by butyryl chloride to provide the phenyl thiobutyryl intermediates of Formula 3.

Scheme 2 shows methods for making the Radiolabeled Arylsulfonyl Compounds of Formula (Ia) from phenyl thiobutyryl compounds of Formula 3 or phenyl thio compounds of Formula 4.

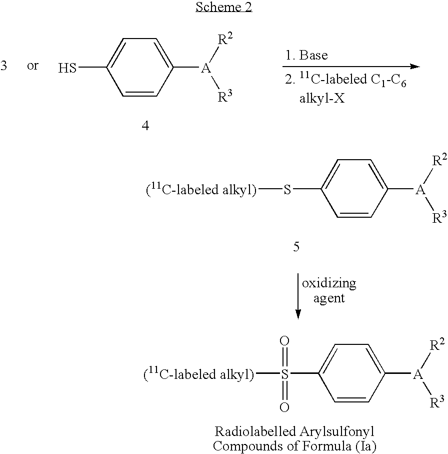

wherein X is a leaving group such as —Cl, —Br, —I, —O-mesyl, —O-tosyl, or —O-triflate; and A, R² and R³ are defined above for the Compounds of Formula (I).

The phenyl thiobutyryl compounds of Formula 3, or alternatively, the phenylthio compounds of Formula 4 can be reacted with a compound of the formula ¹¹C-labeled C₁-C₆ alkyl-X in the presence of a base such as tetrabutylammonium hydroxide or pyrrolidine, to provide the ¹¹C-labeled phenyl thio compounds of Formula 5. The compounds of Formula 5 can then be oxidized using an oxidizing agent, such as potassium peroxymonosulfate, to provide the Radiolabeled Arylsulfonyl Compounds of Formula (Ia).

Scheme 4 shows methods for making the Radiolabeled Arylsulfonyl Compounds of Formula (I) wherein R¹ is a ¹⁸F-labeled C₁-C₆ alkyl group.

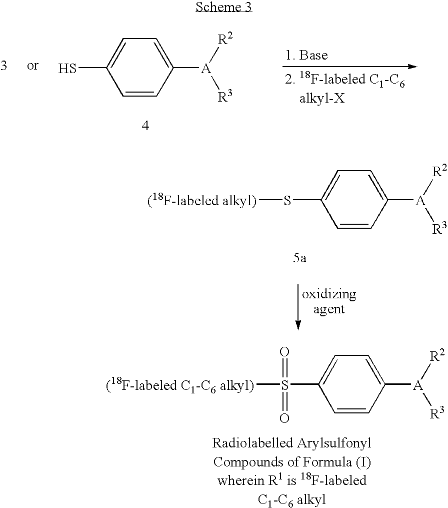

wherein X is a leaving group such as —Cl, —Br, —I, —O-mesyl, —O-tosyl, or —O-triflate; and A, R² and R³ are defined above for the Compounds of Formula (I).

The phenyl thiobutyryl compounds of Formula 3, or alternatively, the phenylthio compounds of Formula 4 can be reacted with a compound of the formula ¹⁸F-labeled C₁-C₆ alkyl-X (which can be made according to methods set forth in Iwata et al., Appl. Rad. Isotopes, 57:347-352 (2002); and Bergman et al., Appl. Rad. Iostopes, 54:923-933 (2001)) in the presence of a base such as tetrabutylammonium hydroxide or pyrrolidine, to provide the ¹⁸F-labeled phenyl thio compounds of Formula 5a. The compounds of Formula 5a can then be oxidized using an oxidizing agent, such as potassium peroxymonosulfate, to provide the Radiolabeled Arylsulfonyl Compounds of Formula (I) wherein R¹ is a ¹⁸F-labeled C₁-C₆ alkyl group.

Scheme 4 shows methods for making the Radiolabeled Arylsulfonyl Compounds of Formula (I) wherein R¹ is a ³H-labeled C₁-C₆ alkyl group.

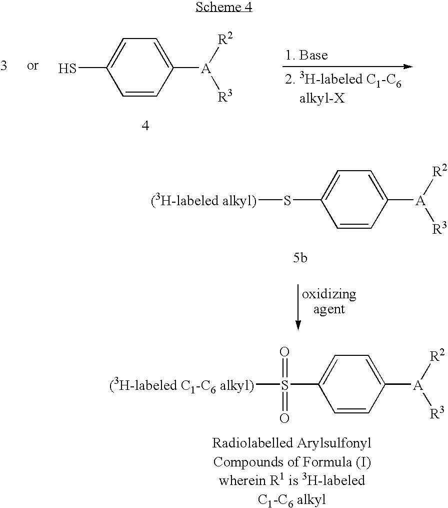

wherein X is a leaving group such as —Cl, —Br, —I, —O-mesyl, —O-tosyl, or —O-triflate; and A, R² and R³ are defined above for the Compounds of Formula (I).

The phenyl thiobutyryl compounds of Formula 3, or alternatively, the phenylthio compounds of Formula 4 can be reacted with a compound of the formula ³H-labeled C₁-C₆ alkyl-X (which may be commercially available or made according to methods well-known to one of ordinary skill in the art of organic chemistry) in the presence of a base such as tetrabutylammonium hydroxide or pyrrolidine, to provide the ³H-labeled phenyl thio compounds of Formula 5b. The compounds of Formula 5b can then be oxidized using an oxidizing agent, such as potassium peroxymonosulfate, to provide the Radiolabeled Arylsulfonyl Compounds of Formula (I) wherein R¹ is a ³H-labeled C₁-C₆ alkyl group.

In another embodiment, illustrated above in Scheme 2, the Radiolabeled Arylsulfonyl Compounds of Formula (Ia) can be made by a method comprising the steps (a), (b), and (c) as described below.

(a) contacting a compound of Formula 3 or formula 4 with a base for a time and at a temperature sufficient to make a compound of Formula 5.

In one embodiment, about 0.5 to about 20 equivalents of the base are used per about 1 equivalent of a compound of Formula 3 or formula 4.

In another embodiment, about 1 to about 10 equivalents of the base are used per about 1 equivalent of a compound of Formula 3 or formula 4.

In another embodiment, about 2 to about 5 equivalents of the base are used per about 1 equivalent of a compound of Formula 3 or formula 4.

Suitable bases for use in the method of step (a) are organic bases such as tetrabutylammonium hydroxide, pyrrolidine, lithium diisopropylamide, lithium diethylamide, sodium methoxide, n-butyllithium, lithium hexamethyldisilazide, sodium hexamethyldisilazide, potassium hexamethyldisilazide, potassium tert-butoxide, piperidine, morpholine, diethylamine, tetramethylpiperidine, diisopropylamine, and triethylamine; and inorganic bases such as sodium hydroxide, potassium hydroxide, potassium carbonate, potassium bicarbonate, and sodium hydride.

In one embodiment, the base is tetrabutylammonium hydroxide.

In another embodiment, the base is pyrrolidine.

The method of step (a) can be carried out in the presence of a polar aprotic solvent, such as THF, DMF, acetone, acetonitrile, DMSO, HMPA, tetramethylene sulfone, 1,4-dioxane, methyl ethyl ketone, ethyl acetate, or mixtures thereof.

In one embodiment, the solvent is THF.

In another embodiment, the solvent is DMF.

In another embodiment, the solvent is substantially anhydrous, i.e., comprises less than about 1% water.

In one embodiment, the method of step (a) is carried out for a time of about 30 seconds to about 10 minutes.

In another embodiment, the method of step (a) is carried out for a time of about 1 minute to about 5 minutes.

In another embodiment, the method of step (a) is carried out for a time of about 2 minute to about 3 minutes.

In one embodiment, the method of step (a) is carried out at a temperature of about −20° C. to about 100° C.

In another embodiment, the method of step (a) is carried out at a temperature of about 0° C. to about 50° C.

In another embodiment, the method of step (a) is carried out at a temperature of about 10° C. to about 30° C.

(b) contacting the product formed in step (a) with a compound of Formula ¹¹C-labeled C₁-C₆ alkyl-X for a time and at a temperature sufficient to make a compound of Formula (III).

In one embodiment, the compound of Formula ¹¹C-labeled C₁-C₆ alkyl-X is ¹¹CH₃I.

The method of step (b) can be carried out in the presence of a polar aprotic solvent, such as THF, DMF, acetone, acetonitrile, DMSO, HMPA, tetramethylene sulfone, 1,4-dioxane, methyl ethyl ketone, ethyl acetate, or mixtures thereof.

In one embodiment, the solvent is THF.

In another embodiment, the solvent is DMF.

In another embodiment, the solvent is substantially anhydrous, i.e., comprises less than about 1% water.

In one embodiment, the method of step (b) is carried out for a time of about 5 minutes to about 2 hours.

In another embodiment, the method of step (b) is carried out for a time of about 30 minutes to about 1 hour.

In one embodiment, the method of step (b) is carried out at a temperature of about −20° C. to about 60° C.

In another embodiment, the method of step (b) is carried out at a temperature of about 0° C. to about 40° C.

In another embodiment, the method of step (b) is carried out at a temperature of about 20° C. to about 30° C.

(c) contacting the product formed in step (c) with an oxidizing agent for a time and at a temperature sufficient to make a Radiolabeled Arylsulfonyl Compound of Formula (Ia).

In one embodiment, about 0.5 to about 20 equivalents of the oxidizing agent are used per about 1 equivalent of a compound of Formula 3 or formula 4.

In another embodiment, about 1 to about 10 equivalents of the oxidizing agent are used per about 1 equivalent of a compound of Formula 3 or formula 4.

In another embodiment, about 2 to about 5 equivalents of the oxidizing agent are used per about 1 equivalent of a compound of Formula 3 or formula 4.

Suitable oxidizing agents for use in the method of step (c) are potassium peroxymonosulfate, Oxone®, hydrogen peroxide, NaIO₄, t-BuOCl, Ca(OCl)₂, NaClO₂, NaOCl, dioxiranes, sodium perborate, KMnO₄ and organic peroxyacids, such as m-chloroperbenzoic acid.

In one embodiment, the oxidizing agent is potassium peroxymonosulfate.

In a specific embodiment, the oxidizing agent is Oxone®.

The method of step (b) can be carried out in the presence of a solvent, including water; organic alcohols such as methanol, ethanol, isopropanol and t-butanol; ethers such as diethyl ether and diphenyl ether; THF, 1,4-dioxane, or mixtures thereof.

In one embodiment, the solvent is a mixture of an organic alcohol and water.

In a specific embodiment, the solvent is aqueous methanol.

In another embodiment, the solvent is substantially anhydrous, i.e., comprises less than about 1% water.

In one embodiment, the method of step (c) is carried out for a time of about 30 seconds to about 10 minutes.

In another embodiment, the method of step (c) is carried out for a time of about 1 minute to about 5 minutes.

In another embodiment, the method of step (c) is carried out for a time of about 2 minute to about 3 minutes.

In one embodiment, the method of step (c) is carried out at a temperature of about −0° C. to about 100° C.

In another embodiment, the method of step (c) is carried out at a temperature of about 25° C. to about 80° C.

In another embodiment, the method of step (c) is carried out at a temperature of about 50° C. to about 70° C.

Radiolabeled Arylsulfonyl Compounds of Formula (Ia) that can be made using the methods of the invention include the compounds listed below:

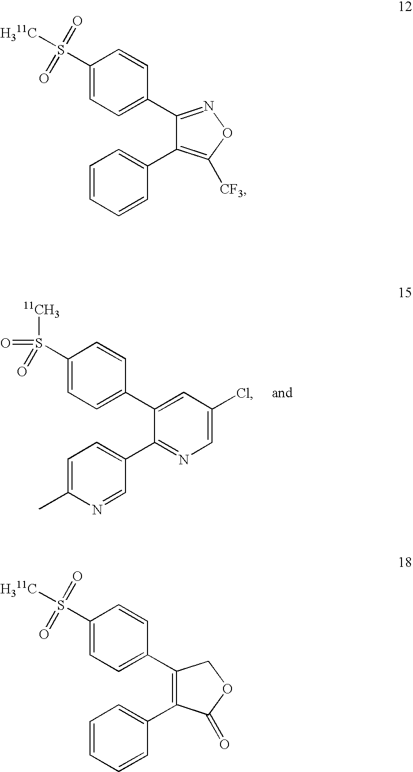

and pharmaceutically acceptable salts thereof.

### Uses of the Radiolabeled Arylsulfonyl Compounds

The Radiolabeled Arylsulfonyl Compounds can be used as imaging agents to image COX-2 expression in a subject.

In one embodiment, the present invention relates to the use of Radiolabeled Arylsulfonyl Compounds for detecting COX-2 expression in vivo. In particular, the present methods for detecting COX-2 expression in vivo contemplate the use of PET, where the imaging probe is a Radiolabeled Arylsulfonyl Compound of the present invention. Further, the present invention provides methods for making phenyl compounds that are radiolabeled at a methylsulfonyl group.

Methods for detecting COX-2 expression in vivo are desired in order to screen individuals for diseases, disorders, states or conditions that are related to COX-2 expression. For example, the following list of processes, diseases or disorders may involve the upregulation of COX-2 protein expression: inflammation, pain, fever, arthritis, Alzheimer's disease, Parkinson's disease, angiogenesis, cancer, ovulation, pregnancy, child birth, renal function, tissue repair, bone metabolism, stroke, myocardial infarction, atherosclerosis, diabetes, allograft rejection, and urogenital disease. Further, radiolabeled COX-2 selective agents can be used to screen for individuals who are more susceptible to side effects of COX-2 inhibitors, as manifested by an increased detection of radiolabeled COX-2 selective agents in specified tissue compartments.

In another embodiment, the invention provides a method for imaging the COX-2 protein in a subject comprising the steps: (a) administering to the subject an imaging-effective amount of a compound having the formula:

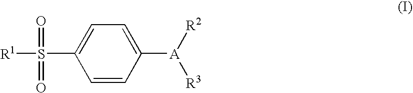

or a pharmaceutically acceptable salt thereof,
wherein:

A is -aryl, —C₃-C₇ cycloalkyl, —C₃-C₇ cycloalkenyl, or -3- to 7-membered heterocycle;

R¹ is a ¹¹C-labeled C₁-C₆ alkyl group, a ¹⁸F-labeled C₁-C₆ alkyl group or a ³H-labeled C₁-C₆ alkyl group;

R² is -aryl, —C₃-C₇ cycloalkyl, —C₃-C₇ cycloalkenyl, or -3- to 7-membered heterocycle, each of which may be unsubstituted or independently substituted with one or more -halo, —CF₃, —C₁-C₆ alkyl, —C₁-C₆ alkenyl, —(C₁-C₆ alkylene)-aryl, —C₁-C₆ alkynyl, —N(R⁴)₂, —CN, —OR⁴, —SR⁴, —S(O)—R⁴, —SO₂—R⁴, —SO₂NH—R⁴, —SO₃H, —NH—SO₂—R⁴, —C(O)R⁵ or —NHC(O)R⁵ groups;

R³ is —H, -halo, —CF₃, —C₁-C₆ alkyl, —C₁-C₆ alkenyl, -aryl, —(C₁-C₆ alkylene)-aryl, —C₁-C₆ alkynyl, —C₃-C₇ cycloalkyl, —C₃-C₇ cycloalkenyl, -3- to 7-membered heterocycle, —N(R⁴)₂, —CN, —OR⁴, —SR⁴, —S(O)—R⁴, —SO₂—R⁴, —SO₂NH—R⁴, —SO₃H, —NH—SO₂—R⁴, —C(O)R⁵ or —NHC(O)R⁵, wherein a —C₃-C₇ cycloalkyl, —C₃-C₇ cycloalkenyl, -3- to 7-membered heterocycle, or -aryl group may be unsubstituted or independently substituted with one or more -halo, —CF₃, —C₁-C₆ alkyl, —C₁-C₆ alkenyl, —(C₁-C₆ alkylene)-aryl, —C₁-C₆ alkynyl, —N(R⁴)₂, —CN, —OR⁴, —SR⁴, —S(O)—R⁴, —SO₂—R⁴, —SO₂NH—R⁴, —SO₃H, —NH—SO₂—R⁴, —C(O)R⁵ or —NHC(O)R⁵ groups;

each R⁴ is independently —H, —C₁-C₆ alkyl, C₁-C₆ alkenyl, —C₁-C₆ alkynyl, -aryl, —(C₁-C₆ alkylene)-aryl, —C₃-C₇ cycloalkyl, —C₃-C₇ cycloalkenyl or -3- to 7-membered heterocycle; and

R⁵ is —R⁴, —N(R⁴)₂ or —OR⁴; and

(b) detecting the radioactive emission of the compound administered in step (a).

In one embodiment, the detecting of step (b) is carried out using PET.

In one embodiment, the Radiolabeled Arylsulfonyl Compounds have high specific activity. In one embodiment, the invention provides Radiolabeled Arylsulfonyl Compounds having a specific activity that is greater than about 1000 Ci/mmol.

In the present methods for detecting COX-2 protein expression in a subject, the step of detecting the ¹¹C and ¹⁸F radioactive emissions of the Radiolabeled Arylsulfonyl Compounds can be conducted using positron emission tomography (PET). PET is useful for visualizing a subject's condition in relation to various tissues, especially bone and soft tissues, such as cartilage, synovium and organs. Specific organs and tissues, include but are not limited to, the brain, colon, joints, heart, kidney, liver, spleen, spinal cord, lymph nodes, or any combination thereof, of the subject. By using PET, a computer tomogram can be obtained of the tissue or organ investigated, enabling the localization and quantification of COX-2 protein. PET imaging can be performed on a subject using the methods described, for example, in McCarthy, T. et al. “Radiosynthesis, in vitro validation, and in vivo evaluation of ¹⁸F-labeled COX-1 and COX-2 inhibitors,” J. Nuclear Med., 43:117-124 (2002).

Further, the RPCs may have a high affinity and specificity to COX-2, as can be reflected in a low IC₅₀ value. In one embodiment, the Radiolabeled Arylsulfonyl Compounds have an IC₅₀ to the COX-2 protein that is from about 0.01 nM to about 200 nM. In other embodiments, the Radiolabeled Arylsulfonyl Compounds have an IC₅₀ to the COX-2 protein that is about 200 nM, 150 nM, 100 nM, 50 nM, 25 nM, 20 nM, 15 nM, 10 nM, 1 nM, 0.5 nM, 0.1 nM, 0.05 nM or 0.01 nM. In another embodiment, the Radiolabeled Arylsulfonyl Compounds have a COX-1/COX-2 IC₅₀ ratio that is from about 100 to about 500,000.

The Radiolabeled Arylsulfonyl Compounds of the present invention can be used to detect and/or quantitatively measure COX-2 protein levels in subjects, including humans. The Radiolabeled Arylsulfonyl Compounds can also be used to measure and/or detect COX-2 protein in COX-2 associated diseases, conditions and disorders, including but not limited to, including arthritis, spondyloarthropathies, systemic lupus erythematosus, autoimmune diseases in general, allograft rejection, asthma, bronchitis, tendinitis, bursitis, skin-related conditions such as psoriasis, eczema, burns and dermatitis, post-operative inflammation including from ophthalmic surgery such as cataract surgery and refractive surgery, gastrointestinal conditions such as inflammatory bowel disease, Crohn's disease, gastritis, irritable bowel syndrome; ulcerative colitis, a neoplastic disease, such as colorectal cancer, and cancer of the breast, lung, prostate, bladder, cervix and skin, vascular diseases, migraine headaches, periarterits nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, neohritiis, hypersensitivity, conjunctivitis, swelling occurring after injury, myocardial ischemia, myochardial infarction, ophthalmic diseases, such as retinitis, retinopathies, uveitis, ocular photophobia, and of acute injury to the eye tissue, allergic rhinitis, respiratory distress syndrome, endotoxin shock syndrome, atherosclerosis, pulmonary inflammation such as from viral and bacterial infections and from cystic fibrosis, central nervous system disorders, such as cortical dementias including Alzheimer's disease, and central nervous system damage resulting from stroke, ischemia and trauma.

The Radiolabeled Arylsulfonyl Compounds can also be used to detect or monitor processes, diseases or disorders that may involve the upregulation of COX-2 protein expression: inflammation, pain, fever, arthritis, Alzheimer's disease, Parkinson's disease, angiogenesis, cancer, ovulation, pregnancy, child birth, renal function, tissue repair, bone metabolism, stroke, myocardial infarction, atherosclerosis, diabetes, allograft rejection, and urogenital disease.

Further, the Radiolabeled Arylsulfonyl Compounds can be used to screen for individuals who are more susceptible to side effects of COX-2 inhibitors, as manifested by an increased detection of the Radiolabeled Arylsulfonyl Compounds in specified tissue compartments.

Additionally, the Radiolabeled Arylsulfonyl Compounds can be used to determine the efficacy of COX-2 inhibitors we administered to a subject to treat a disorder that involves the upregulation of COX-2 protein expression.

Alternatively, the methods for detection can be used to monitor the course of inflammation in an individual. Thus, whether a particular COXIB therapeutic regimen aimed at ameliorating the cause of the inflammatory process, or the inflammatory process itself, is effective, can be determined by measuring the decrease of COX-2 protein expression at suspected sites of inflammation.

### Administration of the Radiolabeled Arylsulfonyl Compounds

Due to their activity, the Radiolabeled Arylsulfonyl Compounds are advantageously useful in veterinary and human medicine. As described above, the Radiolabeled Arylsulfonyl Compounds are useful for imaging COX-2 in a subject.

When administered to a subject, the Radiolabeled Arylsulfonyl Compounds can be administered as a component of a composition that comprises a physiologically acceptable carrier or vehicle. The present compositions, which comprise a Radiolabeled Arylsulfonyl Compound, can be administered orally or by any other convenient route, for example, by infusion or bolus injection, or by absorption through epithelial or mucocutaneous linings (e.g., oral, rectal, and intestinal mucosa, etc.) and can be administered together with another biologically active agent. Administration can be systemic or local. Various delivery systems are known, e.g., encapsulation in liposomes, microparticles, microcapsules, capsules, etc., and can be administered.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result in the release of the Radiolabeled Arylsulfonyl Compounds into the bloodstream. The mode of administration is left to the discretion of the practitioner.

In one embodiment, the Radiolabeled Arylsulfonyl Compounds are administered orally.

In another embodiment, the Radiolabeled Arylsulfonyl Compounds are administered intravenously.

In other embodiments, it can be desirable to administer the Radiolabeled Arylsulfonyl Compounds locally. This can be achieved, for example, and not by way of limitation, by local infusion during surgery, by injection, by means of a catheter, by means of a suppository or enema, or by means of an implant, said implant being of a porous, non-porous, or gelatinous material, including membranes, such as sialastic membranes, or fibers.

In certain embodiments, it can be desirable to introduce the Radiolabeled Arylsulfonyl Compounds into the central nervous system or gastrointestinal tract by any suitable route, including intraventricular, intrathecal, and epidural injection, and enema. Intraventricular injection can be facilitated by an intraventricular catheter, for example, attached to a reservoir, such as an Ommaya reservoir.

Pulmonary administration can also be employed, e.g., by use of an inhaler of nebulizer, and formulation with an aerosolizing agent, or via perfusion in a fluorocarbon or a synthetic pulmonary surfactant. In certain embodiments, the Radiolabeled Arylsulfonyl Compounds can be formulated as a suppository, with traditional binders and excipients such as triglycerides.

In another embodiment the Radiolabeled Arylsulfonyl Compounds can be delivered in a vesicle, in particular a liposome (see Langer, Science 249:1527-1533 (1990) and Treat or prevent et al., Liposomes in the Therapy of Infectious Disease and Cancer 317-327 and 353-365 (1989)).

In yet another embodiment the Radiolabeled Arylsulfonyl Compounds can be delivered in a controlled-release system or sustained-release system (see, e.g., Goodson, in Medical Applications of Controlled Release, supra, vol. 2, pp. 115-138 (1984)). Other controlled or sustained-release systems discussed in the review by Langer, Science 249:1527-1533 (1990) can be used. In one embodiment a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J. Med. 321:574 (1989)). In another embodiment polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

In yet another embodiment a controlled- or sustained-release system can be placed in proximity of a target of the Radiolabeled Arylsulfonyl Compounds, e.g., the spinal column, brain, joints, heart, kidney or gastrointestinal tract, thus requiring only a fraction of the systemic dose.

The present compositions can optionally comprise a suitable amount of a physiologically acceptable excipient so as to provide the form for proper administration to the subject.

Such physiologically acceptable excipients can be liquids, such as water for injection, bactereostatic water for injection, sterile water for injection, and oils, including those of petroleum, subject, vegetable, or synthetic origin, such as peanut oil, soybean oil, mineral oil, sesame oil and the like. The pharmaceutical excipients can be saline, gum acacia; gelatin, starch paste, talc, keratin, colloidal silica, urea and the like. In addition, auxiliary, stabilizing, thickening, lubricating, and coloring agents can be used. In one embodiment the physiologically acceptable excipients are sterile when administered to a subject. Water is a particularly useful excipient when the Radiolabeled Arylsulfonyl Compound is administered intravenously. Saline solutions and aqueous dextrose and glycerol solutions can also be employed as liquid excipients, particularly for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills; pellets, capsules, capsules containing liquids, powders, sustained-release formulations, suppositories, emulsions. aerosols, sprays, suspensions, or any other form suitable for use. In one embodiment the composition is in the form of a capsule (see e.g. U.S. Pat. No. 5,698,155). Other examples of suitable physiologically acceptable excipients are described in Remington's Pharmaceutical Sciences 1447-1676 (Alfonso R. Gennaro eds., 19th ed. 1995), incorporated herein by reference.

In one embodiment the Radiolabeled Arylsulfonyl Compounds are formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment the excipients are of pharmaceutical grade.

In another embodiment the Radiolabeled Arylsulfonyl Compounds can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized-powder or water free concentrate in a hermetically sealed container such as an ampule or sachette indicating the quantity of active agent. Where the Radiolabeled Arylsulfonyl Compounds are to be administered by infusion, they can be dispensed, for example, with an infusion bottle containing sterile pharmaceutical grade water or saline. Where the Radiolabeled Arylsulfonyl Compounds are administered by injection, an ampule of sterile water for injection or saline can be provided so that the ingredients can be mixed prior to administration.

The Radiolabeled Arylsulfonyl Compounds can be administered by controlled-release or sustained-release means or by delivery devices that are well known to those of ordinary skill in the art. Examples include, but arc not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the Radiolabeled Arylsulfonyl Compounds of the invention. The invention thus encompasses single unit dosage forms suitable for oral administration such as, but not limited to, tablets, capsules, gelcaps, and caplets that are adapted for controlled- or sustained-release.

In one embodiment a controlled- or sustained-release composition comprises a minimal amount of a Radiolabeled Arylsulfonyl Compound to image COX-2 protein expression in a subject. Advantages of controlled- or sustained-release compositions include extended activity of the drug, reduced dosage frequency, and increased subject compliance. In addition, controlled- or sustained-release compositions can favorably affect the time of onset of action or other characteristics, such as blood levels of the Radiolabeled Arylsulfonyl Compound, and can thus reduce the occurrence of adverse side effects.

Controlled- or sustained-release compositions can initially release an amount of a Radiolabeled Arylsulfonyl Compound that promptly produces the desired diagnostic effect, and gradually and continually release other amounts of the Radiolabeled Arylsulfonyl Compound to maintain this level of diagnostic effect over an extended period of time. To maintain a constant level of the Radiolabeled Arylsulfonyl Compound in the body, the Radiolabeled Arylsulfonyl Compound can be released from the dosage form at a rate that will replace the amount of Radiolabeled Arylsulfonyl Compound being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions.

The amount of the Radiolabeled Arylsulfonyl Compound that is effective as an imaging agent to detect COX-2 in a subject can be determined using standard clinical and nuclear medicine techniques. In addition, in vitro or in vivo testing can optionally be employed to help identify optimal dosage ranges. The precise dose to be employed will also depend on certain factors—the route of administration, the identity of the subject and the identity of the particular radionuclide being detected- and should be decided according to the judgment of the practitioner and each subject's circumstances in view of, e.g., published clinical studies. Suitable imaging-effective dosage amounts, however, range from about 0.01 mCi to about 30 mCi; about 2 mCi to about 30 mCi; about 10 to about 30 mCi or preferably from about 2 mCi to about 5 mCi. The Radiolabeled Arylsulfonyl Compounds will have a specific activity of >1000 Ci/mmol at the time of administration to insure a low injected mass and adequate counts for imaging. The imaging-effective dosage amounts described herein refer to total amounts administered; that is, if more than one dose of a Radiolabeled Arylsulfonyl Compound is administered, the imaging-effective dosage amounts correspond to the total amount administered.

### Kits

The invention encompasses kits that can simplify the administration of a Radiolabeled Arylsulfonyl Compound to a subject.

A typical kit of the invention comprises a unit dosage form of a Radiolabeled Arylsulfonyl Compound. In one embodiment the unit dosage form is a container, which can be sterile, containing an effective amount of a Radiolabeled Arylsulfonyl Compound and a physiologically acceptable carrier or vehicle. The kit can further comprise a label or printed instructions instructing the use of the Radiolabeled Arylsulfonyl Compound as an imaging agent in order to image COX-2 in a subject.

Kits of the invention can further comprise a device that is useful for administering the unit dosage forms. Examples of such a device include, but are not limited to, a syringe, a drip bag, a patch, an inhaler, and an enema bag.

The following examples are set forth to assist in understanding the invention and should not, of course, be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### EXAMPLES

General Methods: Proton nuclear magnetic resonance (NMR) spectra were obtained from Bruker PPX 300 and 400 MHz spectrophotometer. ¹⁹F NMR spectra were recorded on Bruker PPX 282.5 MHz spectrometer. Spectra are recorded in CDCl₃ and the chemical shifts are reported in parts per million relative to TMS for ¹H NMR and CFCl₃ for ¹⁹F NMR as internal standards. The mass spectra were recorded on JKS-HX 11UHF/HX110 HF Tandem Mass Spectrometer in the FAB+ mode. The HPLC analyses were performed using Waters 1525 HPLC system (column: Phenomenex, Prodigy ODS 4.6×250 mm, 5μ). Flash column chromatography was performed on silica gel (Fisher 200-400 mesh) using the solvent system indicated. The radiochemical and chemical purities were analyzed by RP-HPLC with PDA and NaI detectors.

### Example 1

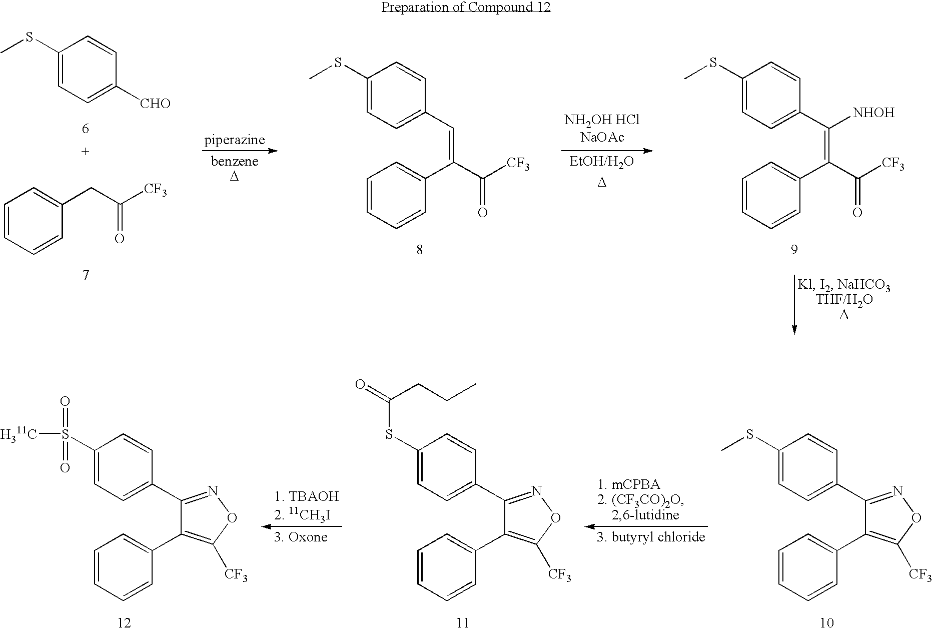

### Synthesis of Compound 10: Compound 10 can be synthesized from Compound 6 as set forth in the scheme above, or alternatively, Compound 10 can be made according to the methods set forth in Habeeb et al., Drug Development Research, 51: 273-286 (2000).

Synthesis of Compound 11: A solution of Compound 10 (80 mg, 0.24 mmol) in CH₂Cl₂ (2 mL) was cooled to about −40° C. and stirred vigorously. A solution of m-CPBA (56 mg of 77% water suspension, 0.25 mmol) in CH₂Cl₂ (2 mL) was then added dropwise. The mixture was stirred at about −20° C. for about 40 min after which Ca(OH)₂ (32 mg, 0.43 mmol) and MgSO₄ (100 mg) were added, and stirring was continued for about 30 min. After filtration and evaporation, the resultant colorless oil was column chromatographed (4% MeOH in CH₂Cl₂) to yield the sulfoxide intermediate as a colorless solid (69 mg, 82%). ¹H NMR: δ 2.73 (s, 3H), 7.21-7.24 (m, 2H), 7.39-7.44 (m, 2H), 7.51-7.62 (m, 5H); ¹⁹F NMR: δ −60.67; HRMS Calcd for C₁₇H₁₃F₃NO₂S (MH⁺): 352.0619; Found: 352.0634.

To a solution of the intermediate sulfoxide (91 mg, 0.26 mmol) in acetonitrile (2 mL), 2,6-lutidine (105 μL, 0.91 mmol) was added, and the mixture was cooled to about −20° C. To the resulting suspension TFAA was added (108 μL, 0.78 mmol) dropwise to give a clear yellow solution. The reaction mixture was then stirred at about −10° C. for about 1 h and then allowed to warm to room temperature. All volatile materials were evaporated under reduced pressure and the residue was dissolved in a precooled (0° C.) mixture of triethylamine (1 mL) and methanol (1 mL). After about 30 min at room temperature, all volatile materials were evaporated at low temperature. The residual yellow oil was dissolved in ethyl ether (5 mL) and extracted with saturated NH₄Cl (6 mL). The layers were separated immediately and the organic layer was dried (MgSO₄) and concentrated at under reduced pressure at about 30° C. to give the crude thiol as a viscous liquid, which could be used for the next step without further purification. A solution of the resultant crude thiol in dichloromethane (1 mL) was treated with pyridine (0.52 mmol, 44 μL) and was cooled to about 0° C. Butyryl chloride (0.39 mmol, 41 μL) was then added to the reaction mixture and was allowed to warm to room temperature over 30 min. The solution was then poured into cold water and extracted with dichloromethane. The combined organic phases were dried over MgSO₄ and concentrated under reduced pressure and column chromatographed (96:4 hexane:EtOAc) to provide Compound 11 as a viscous liquid (44 mg, 43%). ¹H NMR: δ 0.99 (t, J=7.3 Hz, 3H), 1.75 (sextet, J=7.3 Hz, 2H), 2.63 (t, J=7.2 Hz, 2H), 7.23-7.30 (m, 2H), 7.36-7.43 (m, 7H); ¹⁹F NMR: δ −60.67; HRMS Calcd. for C₂₀H₁₇F₃NO₂S (MH⁺): 392.0932; Found: 392.0925.

Synthesis of Compound 12: Compound 11 (1.0 mg) was dissolved in 400 μL of freshly distilled anhydrous THF in a capped 5 mL V-vial. Tetrabutylammonium hydroxide (10 μL, 1M in MeOH) was then added and the resultant pale yellow solution was allowed to stand for 2 minutes. [¹¹C]-Methyl iodide was transported by a stream of argon (20-30 mL/min) into the vial over a period of approximately 5 minutes at room temperature. At the end of the trapping, a suspension of potassium peroxymonosulfate (5 mg) in MeOH:H₂O (1:1 v/v; 200 μL) was introduced into the reaction mixture and was heated on a water bath at 75° C. for about 3-5 minutes. The suspension was filtered through a nylon filter (0.2 μm) and the dark yellow solution was then directly injected into a semi preparative RP-HPLC (Phenomenex C18, 10×250 mm, 10μ) and eluted with acetonitrile: 0.1 M ammonium formate solution (35:65) at a flow rate of 10 mL/min. The precursor appeared at 5-6 minutes during the HPLC analysis. The product fraction with a retention time of 9-10 minutes based on γ-detector was collected, diluted with 100 mL of deionized water, and passed through a classic C-18 Sep-Pak cartridge. Reconstruction of the product in 1 mL of absolute ethanol afforded Compound 12 (50% yield based on ¹¹CH₃I at EOB). A portion of the ethanol solution was analyzed by analytical HPLC (Phenomenex C18; mobile phase: acetonitrile/0.1 M ammonium formate solution 40:60, flow rate: 2 mL/min, retention time: 6.9 min) to determine the specific activity and radiochemical purity.

### Example 2

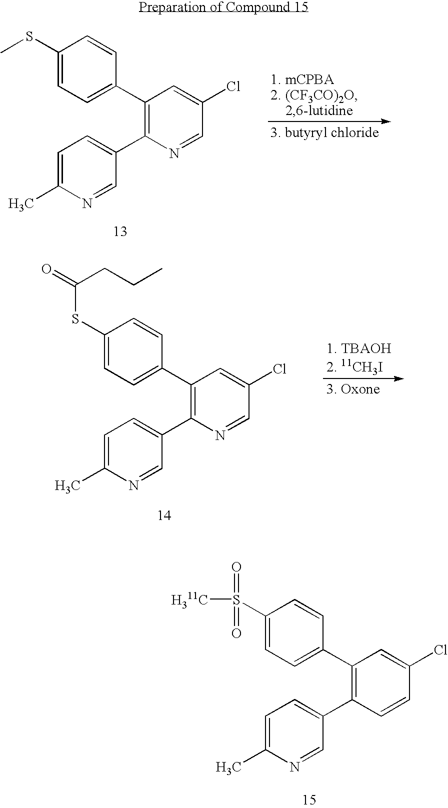

Compound 13 can be synthesized using methodology set forth in Marcoux et al., Organic Letters, 2:2339-2341 (2000).

Synthesis of Compound 14: A solution of Compound 13 (147 mg, 0.45 mmol) in CH₂Cl₂ (2 mL) was cooled to about −40° C. and stirred vigorously. Then a solution of m-CPBA (106 mg of 77% water suspension, 0.47 mmol) in CH₂Cl₂ (2 mL) was added dropwise. The mixture was stirred at about −20° C. for about 40 min. Then Ca(OH)₂ (60 mg, 0.81 mmol) and MgSO₄ (200 mg) were added, and stirring was continued for about 30 min. After filtration and evaporation, the resultant colorless oil was column chromatographed (4% MeOH in CH₂Cl₂) to yield the sulfoxide intermediate as a colorless puffy solid (130 mg, 84%). 14: mp ° C.; ¹H NMR: δ 2.54 (s, 3H), 2.75 (s, 3H), 7.07 (d, J=8.0 Hz, 1H), 7.35-7.37 (m, 2H), 7.56 (dd, J=8.0, 2.3 Hz, 1H), 7.61-7.63 (m, 2H), 7.74 (d, J=2.4 Hz, 1H), 8.42 (d, J=2.1 Hz, 1H), 8.70 (d, J=2.4 Hz, 1H);

HRMS Calcd for C₁₈H₁₆ClN₂OS (MH⁺): 343.0672; Found: 343.0679.

To a solution of the sulfoxide intermediate (50 mg, 0.15 mmol) in acetonitrile (0.75 mL), 2,6-lutidine (60 μL, 0.52 mmol) was added, and the mixture was cooled to about −20° C. To the resulting suspension was added TFAA (60 μL, 0.44 mmol) dropwise to give a clear yellow solution. The reaction mixture was then stirred at about −10° C. for about 1 h and then allowed to warm to room temperature. All volatile materials were evaporated under reduced pressure and the residue was dissolved in a precooled (0° C.) mixture of triethylamine (0.3 mL) and methanol (0.3 mL). After about 30 min at room temperature, all volatile materials were evaporated at low temperature. The residual yellow oil was dissolved in ethyl ether (5 mL) and extracted immediately with saturated NH₄Cl (6 mL). The organic layer was dried (MgSO₄) and concentrated to give the crude thiol as a viscous liquid, which could be used for the next step without further purification. A solution of the resultant crude thiol in dichloromethane (1 mL) was treated with pyridine (0.2918 mmol, 25 μL) and was cooled to about 0° C. Butyryl chloride (0.2189 mmol, 23 μL) was then added to the reaction mixture and was allowed to warm to room temperature over about 30 min. The solution was then poured into cold water and extracted with dichloromethane. The combined organic phases were dried over MgSO₄ and concentrated under reduced pressure and column chromatographed (70:30 hexane:EtOAc) to yield Compound 14 as a viscous liquid (32 mg, 57%). ¹H NMR: δ 1.00 (t, J=7.4 Hz, 3H), 1.75 (sextet, J=7.4 Hz, 2H), 2.53 (s, 3H), 2.65 (t, J=7.4 Hz, 2H), 7.05 (d, J=7.9 Hz, 1H), 7.20-7.22 (m, 2H), 7.36-7.38 (m, 2H), 7.53 (dd, J=8.0, 2.3 Hz, 1H), 7.75 (d, J=2.4 Hz, 1H), 8.46 (d, J=2.0 Hz, 1H), 8.67 (d, J=2.4 Hz, 1H), HRMS Calcd for C₂₁H₂₀CIN₂OS (MH⁺): 383.0985; Found: 383.0992.

Synthesis of Compound 15: Compound 14 (1.0 mg) was dissolved in 400 μL of freshly distilled anhydrous THF in a capped 5 mL V-vial. Tetrabutylammonium hydroxide (10 μL, 1M in MeOH) was then added and the resultant pale yellow solution was allowed to stand for about 2 minutes. [¹¹C]-Methyl iodide was transported by a stream of argon (20-30 mL/min) into the vial over a period of approximately 5 minutes at room temperature. At the end of the trapping, a suspension of potassium peroxymonosulfate (5 mg) in MeOH:H₂O (1:1 v/v; 200 μL) was introduced into the reaction mixture and was heated on a water bath at about 75° C. for about 5 minutes. The suspension was filtered through a nylon filter and the dark yellow solution was then directly injected into a semi preparative RP-HPLC (Phenomenex C18, 10×250 mm, 10μ) and eluted with acetonitrile: 0.1 M ammonium formate solution (35:65) at a flow rate of 10 mL/min. The precursor appeared at 5-6 minutes during the HPLC analysis. The product fraction with a retention time of 9-10 minutes based on γ-detector was collected, diluted with 100 mL of deionized water, and passed through a classic C-18 Sep-Pak cartridge. Reconstruction of the product in 1 mL of absolute ethanol provided Compound 15 (50% yield based on ¹¹CH₃I at EOB).

### Example 3

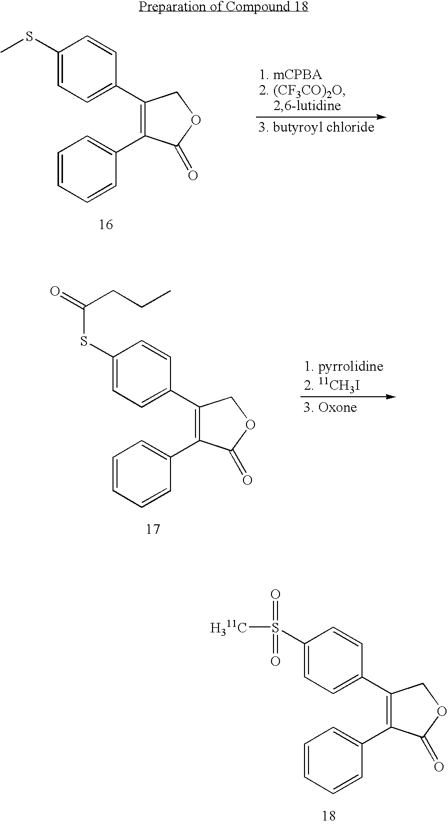

Compound 16 can be synthesized using the methodology set forth in Zhang et al., Organic Letters, 4:4559-4561 (2002).

Synthesis of Compound 17: A solution of Compound 16 (190 mg, 0.67 mmol) in CH₂Cl₂ (5 mL) was cooled to −40° C. and stirred vigorously. Then a solution of mCPBA (145 mg of 77% water suspension, 0.67 mmol) in CH₂Cl₂ (2 mL) was added dropwise. The mixture was stirred at about −20° C. for about 30 min. Then Ca(OH)₂ (89 mg, 1.2 mmol) and MgSO₄ (200 mg) were added, and stirring was continued for about 30 min. After filtration and evaporation, the resultant colorless oil was column chromatographed (3% MeOH in CHCl₃) and triturated from diethylether to yield the sulfoxide intermediate as a colorless solid (185 mg, 93%). ¹H NMR (400 MHz, CDCl₃): δ 2.75 (s, 3H), 5.20 (s, 2H), 7.38-7.43 (m, 5H), 7.48 (d, J=8.4 Hz, 2H), 7.63 (d, J=8.4 Hz, 2H); HRMS Calcd for C₁₇H₁₅O₃S (MH⁺): 299.0742; Found: 299.0747.

To a solution of the sulfoxide intermediate (99 mg, 0.33 mmol) in acetonitrile (2 mL), 2,6-lutidine (135 μL, 1.16 mmol) was added, and the mixture was cooled to about −20° C. To the resulting suspension was added TFAA (138 μL, 0.99 mmol) dropwise to give a clear yellow solution. The reaction mixture was then stirred at about −10° C. for about 1 h and then allowed to warm to room temperature. All volatile materials were evaporated under reduced pressure and the residue was dissolved in a precooled (about 0° C.) mixture of triethylamine (1 mL) and methanol (1 mL). After about 30 min at room temperature, all volatile materials were evaporated at low temperature. The residual yellow oil was dissolved in ethyl ether (5 mL) and extracted with saturated NH₄Cl (6 mL). The layers were separated, and the organic layer was dried (MgSO₄) and concentrated to give the crude thiol as a viscous liquid, which could be used for the next step without further purification.

A solution of the resultant crude thiol in dichloromethane (1 mL) was treated with pyridine (54 μL, 0.66 mmol) and was cooled to about 0° C. Butyryl chloride (52 μL, 0.50 mmol) was then added to the reaction mixture and was allowed to warm to room temperature over about 30 min. The solution was then poured into cold water and extracted with dichloromethane. The combined organic phases were dried over MgSO₄ and concentrated under reduced pressure and column chromatographed (85:15 hexane:EtOAc) to yield Compound 17 as a viscous liquid (61 mg, 54%). ¹H NMR (400 MHz, CDCl₃): δ 0.99 (t, J=7.4 Hz, 3H), 1.75 (sextet, J=7.4 Hz, 2H), 2.65 (t, J=7.3 Hz, 2H), 5.18 (s, 2H), 7.32-7.36 (m, 2H), 7.38-7.45 (m, 7H); HRMS Calcd for C₂₀H₁₉O₃S (MH⁺): 339.1055; Found: 339.1068.

Synthesis of Compound 18: Compound 17 (1.0 mg) was dissolved in 400 μL of DMF in a capped 5 mL V-vial. Pyrrolidine (10 μL) was then added and the resultant pale yellow solution was allowed to stand for 2 minutes. [¹¹C]-Methyl iodide was transported by a stream of argon (20-30 mL/min) into the vial over a period of approximately 5 minutes at room temperature. At the end of the trapping, a suspension of potassium peroxymonosulfate (5 mg) in MeOH:H₂O:THF (1:1:1 v/v; 600 μL) was introduced into the reaction mixture and was heated on a water bath at about 75° C. for 3-5 minutes. The suspension was filtered through a nylon filter and the dark yellow solution was then directly injected into a semi preparative RP-HPLC (Phenomenex C18, 10×250 mm, 10μ) and eluted with acetonitrile: 0.1 M ammonium formate solution (30:70) at a flow rate of 10 mL/min. The product fraction with a retention time of 9-10 minutes based on γ-detector was collected, diluted with 100 mL of deionized water, and passed through a classic C-18 Sep-Pak cartridge. Reconstruction of the product in 1 mL of absolute ethanol provided Compound 18 (50% yield based on ¹¹CH₃I at EOB).

What is claimed:

1. A method for detecting cyclooxygenase-2 protein expression in a subject in vivo, the method comprising the steps:
(a) administering to the subject an imaging-effective amount of a compound having the formula: 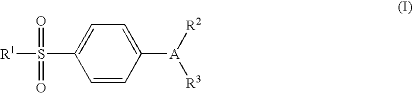
or a pharmaceutically acceptable salt thereof,
wherein:
A is -aryl, —C₃-C₇ cycloalkyl, —C₃-C₇ cycloalkenyl, or -3- to 7-membered heterocycle;
R¹ is a ¹¹C-labeled C₁-C₆ alkyl group, a ¹⁸F-labeled C₁-C₆ alkyl group or a ³H-labeled C₁-C₆ alkyl group;
R² is -aryl, —C₃-C₇ cycloalkyl, —C₃-C₇ cycloalkenyl, or -3- to 7-membered heterocycle, each of which may be unsubstituted or independently substituted with one or more -halo, —CF₃, —C₁-C₆ alkyl, —C₁-C₆ alkenyl, —(C₁-C₆ alkylene)-aryl, —C₁-C₆ alkynyl, —N(R⁴)₂, —CN, —OR⁴, —SR⁴, —S(O)—R⁴, —SO₂—R⁴, —SO₂NH—R⁴, —SO₃H, —NH—SO₂—R⁴, —C(O)R⁵ or —NHC(O)R⁵ groups;
R³ is —H, -halo, —CF₃, —C₁-C₆ alkyl, —C₁-C₆ alkenyl, -aryl, —(C₁-C₆ alkylene)-aryl, —C₁-C₆ alkynyl, —C₃-C₇ cycloalkyl, —C₃-C₇ cycloalkenyl, -3- to 7-membered heterocycle, —N(R⁴)₂, —CN, —OR⁴, —SR⁴, —S(O)—R⁴, —SO₂—R⁴, —SO₂NH—R⁴, —SO₃H, —NH—SO₂—R⁴, —C(O)R⁵ or —NHC(O)R⁵, wherein a —C₃-C₇ cycloalkyl, —C₃-C₇ cycloalkenyl, -3- to 7-membered heterocycle, or -aryl group may be unsubstituted or independently substituted with one or more -halo, —CF₃, —C₁-C₆ alkyl, —C₁-C₆ alkenyl, —(C₁-C₆ alkylene)-aryl, —C₁-C₆ alkynyl, —N(R⁴)₂, —CN, —OR⁴, —SR⁴, —S(O)—R⁴, —SO₂—R⁴, —SO₂NH—R⁴, —SO₃H, —NH—SO₂—R⁴, —C(O)R⁵ or —NHC(O)R⁵ groups;
each R⁴ is independently —H, —C₁-C₆ alkyl, C₁-C₆ alkenyl, —C₁-C₆ alkynyl, -aryl, —(C₁-C₆ alkylene)-aryl, —C₃-C₇ cycloalkyl, —C₃-C₇ cycloalkenyl or -3- to 7-membered heterocycle; and
R⁵ is —R⁴, —N(R⁴)₂ or —OR⁴; and
(b) detecting the radioactive emission of the compound administered in step (a).

2. The method of claim 1, wherein for the compound of Formula (I), A is -aryl or -3- to 7-membered heterocycle.

3. The method of claim 2 wherein A is -phenyl, -isoxazolyl, -pyridyl, or -dihydrofuran-2-one.

4. The method of claim 1, wherein for the compound of Formula (I), R² is -aryl or -3- to 7-membered heterocycle.

5. The method of claim 4 wherein R² is -phenyl or -pyridyl.

6. The method of claim 1, wherein for the compound of Formula (I), R³ is —H, -halo, —C₁-C₆ alkyl, or —CF₃.

7. The method of claim 1, wherein for the compound of Formula (I), R¹ is a ¹¹C-labeled C₁-C₆ alkyl group.

8. The method of claim 7, wherein R¹ is —¹¹CH₃.

9. The method of claim 8, wherein the compound of Formula (I) is: 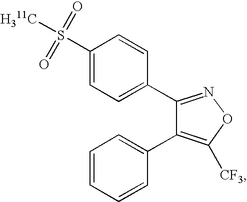
or a pharmaceutically acceptable salt thereof.

10. The method of claim 8, wherein the compound of Formula (I) is: 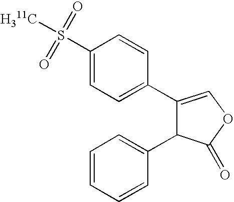
or a pharmaceutically acceptable salt thereof.

11. The method of claim 8, wherein the compound of Formula (I) is: 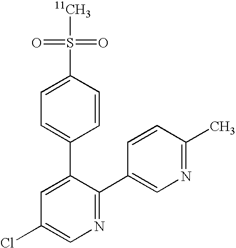
or a pharmaceutically acceptable salt thereof.

12. The method of claim 1, wherein in step (b) the radioactive emission is detected using positron-emission tomography.

13. The method of claim 1, wherein in step (b) the radioactive emission is detected in the brain, joints, heart, kidney or any combination thereof, of the subject.

14. The method of claim 1, wherein the subject is known or suspected to have one or more of the following conditions: an inflammatory disorder, arthritis, a neoplastic disease, atherosclerosis, stroke, myocardial infarction, diabetes, allograft rejection, a urogenital disease, a central nervous system disorder, a brain injury, a brain disorder or a renal disorder.

15. The method of claim 14 wherein the neoplastic disease is cancer.

16. The method of claim 14 wherein the central nervous system disorder is Alzheimer's disease or Parkinson's disease.

17. The method of claim 1, wherein the compound of Formula (I) has an IC₅₀ to the cyclooxygenase-2 protein that is about 200 nM, 150 nM, 100 nM, 50 nM, 25 nM, 20 nM, 15 nM, 10 nM or 5 nM.

18. The method of claim 1, wherein the compound of Formula (I) has an cyclooxygenase-1/cyclooxygenase-2 IC₅₀ ratio that is greater than about 1500 nM.

19. The method of claim 1, wherein the compound of Formula (I) has a specific activity that is greater than about 1000 Ci/mmol.

20. A method for making a compound having the formula: 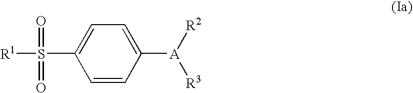
wherein:
A is -aryl, —C₃-C₇ cycloalkyl, —C₃-C₇ cycloalkenyl, or -3- to 7-membered heterocycle;
R¹ is a ¹¹C-labeled C₁-C₆ alkyl group;
R² is -aryl, —C₃-C₇ cycloalkyl, —C₃-C₇ cycloalkenyl, or -3- to 7-membered heterocycle, each of which may be unsubstituted or independently substituted with one or more -halo, —CF₃, —C₁-C₆ alkyl, —C₁-C₆ alkenyl, —(C₁-C₆ alkylene)-aryl, —C₁-C₆ alkynyl, —N(R⁴)₂, —CN, —OR⁴, —SR⁴, —S(O)—R⁴, —SO₂—R⁴, —SO₂NH—R⁴, —SO₃H, —NH—SO₂—R⁴, —C(O)R⁵ or —NHC(O)R⁵ groups;
R³ is —H, -halo, —CF₃, —C₁-C₆ alkyl, —C₁-C₆ alkenyl, -aryl, —(C₁-C₆ alkylene)-aryl, —C₁-C₆ alkynyl, —C₃-C₇ cycloalkyl, —C₃-C₇ cycloalkenyl, -3- to 7-membered heterocycle, —N(R⁴)₂, —CN, —OR⁴, —SR⁴, —S(O)—R⁴, —SO₂—R⁴, —SO₂NH—R⁴, —SO₃H, —NH—SO₂—R⁴, —C(O)R⁵ or —NHC(O)R⁵, wherein a —C₃-C₇ cycloalkyl, —C₃-C₇ cycloalkenyl, -3- to 7-membered heterocycle, or -aryl group may be unsubstituted or independently substituted with one or more -halo, —CF₃, —C₁-C₆ alkyl, —C₁-C₆ alkenyl, —(C₁-C₆ alkylene)-aryl, —C₁-C₆ alkynyl, —N(R⁴)₂, —CN, —OR⁴, —SR⁴, —S(O)—R⁴, —SO₂—R⁴, —SO₂NH—R⁴, —SO₃H, —NH—SO₂—R⁴, —C(O)R⁵ or —NHC(O)R⁵ groups;
each R⁴ is independently —H, —C₁-C₆ alkyl, C₁-C₆ alkenyl, —C₁-C₆ alkynyl, -aryl, —(C₁-C₆ alkylene)-aryl, —C₃-C₇ cycloalkyl, —C₃-C₇ cycloalkenyl or -3- to 7-membered heterocycle; and
R⁵ is —R⁴, —N(R⁴)₂ or —OR⁴,
the method comprising the steps:
(a) contacting a compound having the formula: 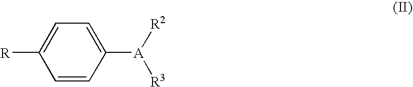
wherein:
R is —SH or —SC(O)(CH₂)₂CH₃ and A, R² and R³ are as defined above for the compounds of formula (Ia), with a base for a time and temperature sufficient to make the compound having the formula (III): 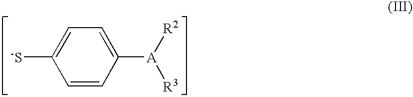
wherein A, R² and R³ are as defined above for the compounds of Formula (Ia);
(b) contacting the compound of formula (III) with a compound having the formula R¹—X for a time and at a temperature sufficient to make a compound of Formula (IV): 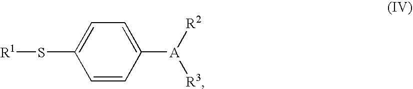
wherein R¹, A, R² and R³ are as defined above for the compounds of Formula (Ia); and
(c) contacting the compound of Formula (III) with an oxidizing agent for a time and temperature sufficient to make a compound having the formula (Ia): 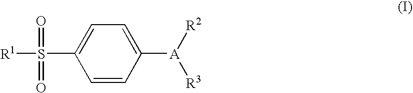
wherein A, R¹, R² and R³ are as defined above for the compounds of Formula (Ia).

21. The method of claim 20 wherein, the compound of Formula (Ia) is: 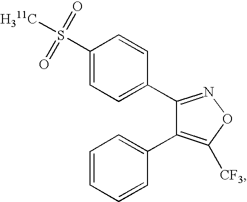
and the compound of Formula (II) is: 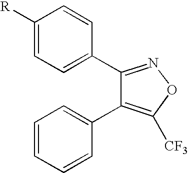
wherein R is —SH or —SC(O)CH₂CH₂CH₃.

22. The method of claim 20 wherein, the compound of Formula (Ia) is: 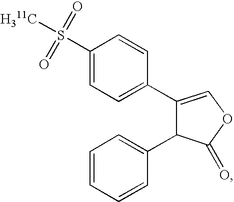
and the compound of Formula (II) is: 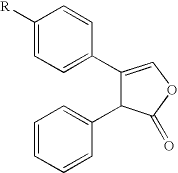
wherein R is —SH or —SC(O)CH₂CH₂CH₃.

23. The method of claim 20 wherein, the compound of Formula (Ia) is: 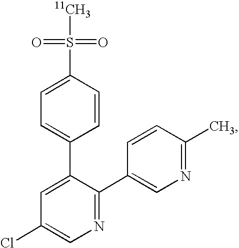
and the compound of Formula (II) is: 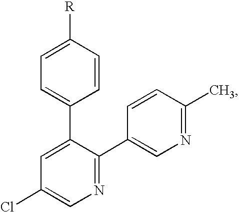
wherein R is —SH or —SC(O)CH₂CH₂CH₃.

24. The method of claim 20, further comprising the step of isolating the product obtained in step (c).

25. The method of claim 20, wherein the contacting of step (a) is conducted at about 70° C. for about 3 minutes.

26. The method of claim 20, wherein the base in step (a) is tetrabutylammonium hydroxide or pyrrolidine.

27. The method of claim 20 wherein in step (b), the compound of formula R¹—X is ¹¹CH₃I.

28. The method of claim 20, wherein in step (c), the oxidizing agent is potassium peroxymonosulfate.

29. The method of claim 28, wherein the oxidizing agent is Oxone®.
